(19) United States
(12) Patent Application Publication
(10) Pub. No.: US 2021/0330398 A1
(43) Pub. Date: Oct. 28, 2021

(54) SINGLE-CORE FIBER AND MULTI-CORE FIBER CONFIGURATIONS FOR MEDICAL DEVICES

(71) Applicant: St. Jude Medical International Holding S.à.r.l., Luxembourg (LU)

(72) Inventors: Troy TEGG, Elk River, MN (US); Greg OLSON, Elk River, MN (US)

(21) Appl. No.: 17/237,023

(22) Filed: Apr. 21, 2021

Related U.S. Application Data
(60) Provisional application No. 63/013,879, filed on Apr. 22, 2020.

(51) Int. Cl. A61B 34/20

(52) U.S. Cl. CPC A61B 34/20; A61B 5/6852; A61B 2034/2061

(57) ABSTRACT

A medical device may include a tubular body defining a central longitudinal axis and a lumen, the tubular body including an annular wall having an inner circumferential surface and an outer circumferential surface. The medical device may include a multi-core fiber extending along at least a portion of the length of the tubular body and at least partially disposed in the annular wall thereof. The medical device may include a plurality of single-core fibers extending along at least a portion of the length of the tubular body and defining a central longitudinal axis that is nonparallel to the central longitudinal axis of the tubular body. A medical device may include a multi-core fiber disposed on a top surface of a first layer and extending along at least a portion of the length of the first layer; and a second layer deposited or printed on the first layer.

### BACKGROUND

Various systems are known for determining the position and orientation of a medical device. Such systems are used by practitioners for visualization and navigation purposes as the medical device is advanced through a patient's body to the intended site.

One such system utilizes a fluoropaque marker (e.g., a metallic coil, an active impedance-sensing electrode, and the like) coupled to the tip of the needle and another sensor wound around the tip of a guidewire inserted through the needle and used to deliver a cardiac rhythm device, replacement heart valve, etc. to the desired location within the patient's body. The sensors are visible when exposed to a field of ionizing radiation (e.g., X-rays used in fluoroscopy). A display outputs a visual representation of the needle and the guidewire inside the patient's body based on the position of the sensors under the radiation. However, these methods require that radiation be used during the entire procedure in order that the sensors generate output indicative of the position of the needle and guidewire throughout the procedure. Accordingly, the physician's hands also must be exposed to radiation during the entire procedure. Even after the needle has been placed, the physician's hands are still exposed to radiation while inserting the guidewire through the needle and navigating the guidewire toward the heart.

Additional techniques for determining position and/or orientation of the catheter include magnetic, electrical, and/or ultrasound techniques. For example, one type of localization system is an electrical impedance-based system. Electrical impedance-based systems generally include one or more pairs of body surface electrodes (e.g., patches) provided outside a patient's body, a reference sensor (e.g., another patch) attached to the patient's body, and one or more sensors (e.g., electrodes) attached to the medical device. The pairs can be adjacent, linearly arranged, or associated with respective axes of a coordinate system for such a positioning system. The system can determine the position and orientation of the medical device by applying a current across pairs of electrodes, measuring respective voltages induced at the device electrodes (i.e., with respect to the reference sensor), and then processing the measured voltages.

Another system is known as a magnetic field-based positioning system. This type of system generally includes one or more magnetic field generators attached to or placed near the patient bed or other component of the operating environment and one or more magnetic field detection coils coupled with a medical device. Alternatively, the field generators may be coupled with a medical device, and the detection coils may be attached to or placed near a component of the operating environment. The generators provide a controlled low-strength AC magnetic field in the area of interest (i.e., an anatomical region). The detection coils produce a respective signal indicative of one or more characteristics of the sensed field. The system then processes these signals to produce one or more position and orientation readings associated with the coils (and thus with the medical device). The position and orientation readings are typically taken with respect to the field generators, and thus the field generators serve as the de facto “origin” of the coordinate system of a magnetic field-based positioning system. Unlike an electrical impedance-based system, where the coordinate system is relative to the patient on which the body surface electrodes are applied, a magnetic field-based system has a coordinate system that is independent of the patient.

Both electrical impedance-based and magnetic field-based positioning systems provide advantages. For example, electrical impedance-based systems provide the ability to simultaneously locate (i.e., provide a position and orientation reading for) a relatively large number of sensors on multiple medical devices. However, because electrical impedance-based systems employ electrical current flow in the human body, the coordinate systems can be non-homogenous, anisotropic, and not orthonormal (i.e., the basic vectors of the coordinate system are not guaranteed to be at right angles to one another or to have proper unit lengths). Additionally, electrical impedance-based systems may be subject to electrical interference. As a result, geometries and representations that are rendered based on position measurements may appear distorted relative to actual images of subject regions of interest. Magnetic field-based coordinate systems, on the other hand, are not dependent on characteristics of the patient's anatomy and provide a generally orthonormal coordinate system. However, magnetic field-based positioning systems are generally limited to tracking relatively fewer sensors.

In addition, technological advances in the art increasingly demand efficient use of available space in medical devices. For example, catheters used in mapping and ablation can be very small in diameter. In some instances, catheters are as small as 2 to 6 French (1 French=0.3 mm), and in others, they can be even smaller. As such, assembly of a catheter, such as connecting wires to the electrode and stringing those wires through the catheter can be difficult. In some instances, due to the difficulty in adhering the wires to the electrodes, defective catheters may be produced, resulting in poor signals, waste and lowered manufacturing efficiency. In addition, incorporating components used to determine the position and orientation of medical devices can occupy space that otherwise would be used by other valuable components, forcing a costly trade-off.

Accordingly, there is a need for improved medical devices that provide position and orientation readings using components that occupy minimal space.

### SUMMARY

According to one aspect, a medical device or a portion thereof may include a tubular body defining a central longitudinal axis and a lumen, the tubular body including an annular wall having an inner circumferential surface and an outer circumferential surface; and a multi-core fiber extending along at least a portion of the length of the tubular body, wherein the multi-core fiber is at least partially disposed in the annular wall of the tubular body.

According to another aspect, a medical device or a portion thereof may include a tubular body defining a central longitudinal axis and a lumen, the tubular body including an annular wall having an inner circumferential surface and an outer circumferential surface; and a plurality of single-core fibers, each single-core fiber extending along at least a portion of the length of the tubular body and defining a central longitudinal axis that is nonparallel to the central longitudinal axis of the tubular body.

According to a further aspect, a medical device or a portion thereof may include a multi-core fiber disposed on a top surface of a first layer and extending along at least a portion of the length of the first layer, wherein the multi-core fiber includes a plurality of fiber cores, each fiber core including one or more fiber Bragg gratings distributed along the length of the fiber core; and a second layer deposited or printed on the first layer.

### BRIEF DESCRIPTION OF THE DRAWINGS

FIG. 1A is an isometric view of a tubular body and an end view of a multi-core fiber, in accordance with one or more embodiments of the invention.

FIGS. 1B through 1G show end views of medical device bodies with different cross-sectional shapes, in accordance with one or more embodiments of the invention.

FIG. 2A is an end view of a multi-core fiber, in accordance with one or more embodiments of the invention.

FIG. 2B is an isometric view of the multi-core fiber depicted in FIG. 2A, in accordance with one or more embodiments of the invention.

FIG. 3A is an end view of a tubular body with a multi-core fiber partially embedded in a wall of the tubular body, in accordance with one or more embodiments of the invention.

FIG. 3B is an isometric view of the tubular body depicted in FIG. 3A showing the multi-core fiber in a parallel configuration, in accordance with one or more embodiments of the invention.

FIGS. 3C-3D are isometric views of the tubular body depicted in FIG. 3A showing the multi-core fiber in various nonparallel configurations, in accordance with one or more embodiments of the invention.

FIG. 4A is an end view of a tubular body with a multi-core fiber completely embedded in a wall of the tubular body, in accordance with one or more embodiments of the invention.

FIG. 4B is an isometric view of the tubular body depicted in FIG. 4A showing the multi-core fiber in a parallel configuration, in accordance with one or more embodiments of the invention.

FIGS. 4C-4D are isometric views of the tubular body depicted in FIG. 4A showing the multi-core fiber in various nonparallel configurations, in accordance with one or more embodiments of the invention.

FIG. 5A is an end view of a tubular body with a plurality of single-core fibers completely embedded in a wall of the tubular body, in accordance with one or more embodiments of the invention.

FIG. 5B is an isometric view of the tubular body depicted in FIG. 5A showing the plurality of single-core fibers in a parallel configuration, in accordance with one or more embodiments of the invention.

FIGS. 5C-5D are isometric views of the tubular body depicted in FIG. 5A showing the plurality of single-core fibers in various nonparallel configurations, in accordance with one or more embodiments of the invention.

FIG. 6A is a side view of a plurality of single-core fibers in a twisted configuration, in accordance with one or more embodiments of the invention.

FIG. 6B is an isometric view of a tubular body showing the plurality of single-core fibers depicted in FIG. 6A embedded in a wall of the tubular body, in accordance with one or more embodiments of the invention.

FIG. 6C is an isometric view of a tubular body showing the plurality of single-core fibers depicted in FIG. 6A disposed in a lumen of the tubular body, in accordance with one or more embodiments of the invention.

FIG. 7A is an isometric view of a multi-layered medical device portion, in accordance with one or more embodiments of the invention.

FIG. 7B is a cross-sectional view of the multi-layered medical device portion depicted in FIG. 7A taken along line 7-7, in accordance with one or more embodiments of the invention.

FIG. 8 is a cross-sectional view of the multi-layered medical device portion depicted in FIG. 7A taken along line 7-7 shown with a channel, in accordance with one or more embodiments of the invention.

FIGS. 9A through 9C are isometric views of various channels formed in a single layer of a medical device portion, in accordance with one or more embodiments of the invention.

### DETAILED DESCRIPTION

The present invention provides various configurations for incorporating optical fibers into medical devices or portions of medical devices. The configurations disclosed herein can be employed to overcome numerous challenges known in the art, such as routing optical fibers around internal components and through the limited internal space of medical devices or portions thereof. For example, some embodiments provide configurations in which the optical fibers can be incorporated into a medical device or a portion of a medical device without occupying any of the internal space of the medical device, freeing up space for other components. Other embodiments provide configurations in which the optical fibers can be incorporated into a medical device or a portion of a medical device in ways that minimize the amount of internal space occupied by the optical fibers. Further embodiments provide configurations in which the optical fibers can be integrated with a medical device or a portion of a medical device using additive processes that simplify the construction and manufacturing thereof.

The medical devices or portions thereof into which the optical fibers can be incorporated can include any interventional or surgical device, or any portion thereof. Examples of medical devices and medical device portions include, without limitation, catheters, sheaths, guidewires, introducers, and any portions thereof. Examples of catheters include, without limitation, ablation catheters, mapping catheters, and the like. Examples of catheter portions include, without limitation, a shaft (e.g., an elongate shaft), a loop (e.g., a loop portion of a circular mapping catheter), or a strut(s), an arm(s), or a spline(s) of an expandable portion (e.g., a basket, an array, a planar end, etc.) of an ablation catheter and/or mapping catheter. More specific examples of medical devices or portions thereof include, without limitation, steerable sheaths, such as the Agilis™ NxT Steerable Introducer (Abbott Laboratories); radiofrequency (RF) ablation catheters, such as the FlexAbility™ Ablation Catheter and FlexAbility™ Ablation Catheter, Sensor Enabled™ (Abbott Laboratories); and/or mapping catheters, such as Advisor™ HD Grid Mapping Catheter, Sensor Enabled™, Advisor™ FL Circular Mapping Catheter, Sensor Enabled™, and Advisor™ VL Mapping Catheter, Sensor Enabled™ (Abbott Laboratories). These shall not be limiting as other medical devices and medical device portions can be utilized herein without departing from the scope of the present invention.

The medical devices and medical device portions can be equipped with optical sensing technologies, such as fiber Bragg grating (FBG) sensors and/or interferometer sensors, which can be utilized to detect the force, shape (e.g., position and/or orientation), and/or temperature of medical devices or at least the portions of medical devices that include the optical sensor. The optical sensors located on the medical device or medical device portion can be configured to receive an optical input via the optical fiber (e.g., via a multi-core fiber or a plurality of single-core fibers), wherein information regarding the force, shape, and/or temperature is determined from light reflected by the sensor. In general, an optical fiber can include one or more fiber cores, wherein each of the fiber cores can include one or more optical sensors located longitudinally along a portion of the fiber core. As mentioned above, one example of an optical sensor is an FBG sensor, which can be utilized for one or more of temperature sensing (e.g., detecting changes in temperature), force sensing (e.g., detecting forces impacting a catheter tip at a distal end in response to contact pressures from body tissue), and shape sensing (e.g., determining the position and/or orientation of all or a portion of a medical device, such as a distal end of an elongate shaft, a catheter tip, etc., during a medical procedure).

Accordingly, embodiments provide medical devices or portions of medical devices that include one or more optical fibers incorporated into a structural member. In general, an optical fiber includes one or more fiber cores, wherein each fiber core can include one or more optical sensors distributed along a length of the fiber core. Embodiments can include a multi-core fiber including a plurality of fiber cores, or a plurality of single-core fibers (e.g., two or more single-core fibers). For example, in some embodiments, a multi-core fiber is incorporated into the structural member of a medical device or medical device portion. In some embodiments, a plurality of single-core fibers (e.g., two or more single-core fibers) are incorporated into the structural member of a medical device or medical device portion. The structural member is not particularly limited and can have any cross-sectional shape. For example, in some embodiments, the structural member is a tubular body. In some embodiments, the structural member is a substrate. In addition, the structural member can have varying lengths and/or degrees of flexibility. For example, in some embodiments, the structural member is elongated. In some embodiments, the structural member is deformable.

In accordance with one or more embodiments, the one or more optical fibers can be arranged in any of a variety of configurations. For example, in some embodiments, one or more optical fibers can extend partially or completely along a length of the structural member. In some embodiments, one or more optical fibers can be partially disposed in the structural member (e.g., a wall of a tubular body). In some embodiments, one or more optical fibers can be completely disposed in the structural member (e.g., a wall of a tubular body). In some embodiments, one or more optical fibers can be partially disposed in an inner space of the structural member (e.g., a lumen of a tubular body). In some embodiments, one or more optical fibers can be completely disposed in an inner space of the structural member (e.g., a lumen of a tubular body). In some embodiments, one or more optical fibers can be disposed between one or more layers (e.g., between a substrate and a first layer).

In accordance with one or more embodiments, the one or more optical fibers can be arranged in a parallel configuration or a nonparallel configuration. A parallel configuration is generally a configuration in which a central longitudinal axis of an optical fiber is parallel to an axis of the structural member. For example, in some embodiments, a central longitudinal axis of an optical fiber is parallel to a central longitudinal axis of a tubular body. In some embodiments, a central longitudinal axis of an optical fiber is parallel to a longitudinal axis of a substrate. A nonparallel configuration is generally a configuration in which a central longitudinal axis of an optical fiber is nonparallel to an axis of the structural member. For example, in some embodiments, an optical fiber defines a central longitudinal axis that is nonparallel to a central longitudinal axis of a tubular body. In some embodiments, a central longitudinal axis of an optical fiber is nonparallel to a longitudinal axis of a substrate. In one embodiment, at least one optical fiber is helically wound about a central longitudinal axis of a tubular body. In another embodiment, at least one optical fiber is disposed in a serpentine configuration in a tubular body or substrate. In a further embodiment, a plurality of optical fibers can be helically twisted together in a twisted configuration.

In accordance with one or more embodiments, the medical devices and portions thereof can comprise additional components including, for example and without limitation, pull wires, planarity wires, fluid irrigation or drainage lumens, lead wires for the ablation elements, rotation wires, conductive traces, dielectric materials, electrodes, and the like.

Now having generally described the medical devices and portions thereof, specific embodiments will now be discussed, with the proviso that any combination of the alternatives and/or variations discussed above and below can be applied to and across all embodiments of the present disclosure.

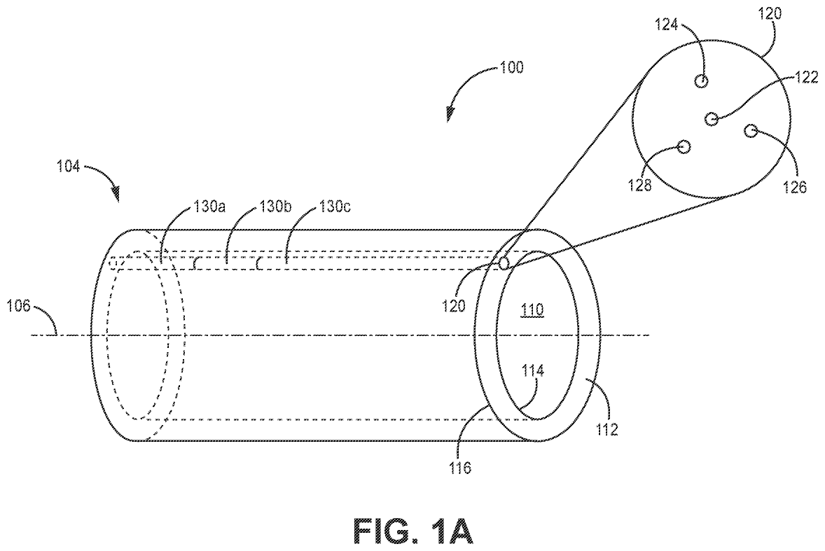

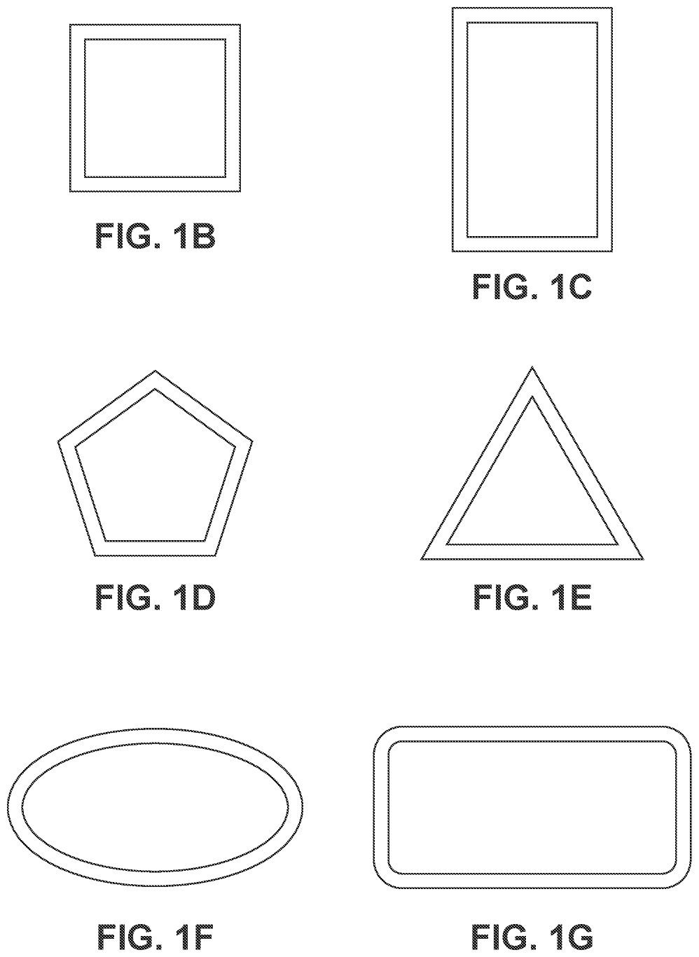

FIG. 1A is an isometric view of a representative portion of a medical device 100, in accordance with an embodiment of the invention. As shown in FIG. 1A, in one embodiment, the medical device portion 100 can include a tubular body 104 and a multi-core fiber 120 extending along at least a portion of the length of the tubular body 104. The tubular body 104 can define a central longitudinal axis 106 and a lumen 110. The tubular body 104 can include an annular wall 112 having an inner circumferential surface 114 and an outer circumferential surface 116. The multi-core fiber 120 can be disposed in the annular wall 112 of the tubular body 104 between the inner circumferential surface 114 and outer circumferential surface 116. In the embodiment shown in FIG. 1A, the multi-core fiber 120 includes a plurality of fiber cores 122, 124, 126, and 128. Each of the plurality of fiber cores 122, 124, 126, and 128 can include one or more optical sensors 130a, 130b, and 130c. In some embodiments, the one or more optical sensors 130a, 130b, and 130c can be located adjacent to one another along a particular fiber core. For example, fiber core 126 can include a plurality of optical sensors 130a, 130b, and 130c distributed along the length of the fiber core 126 (e.g., optical sensors 130a, 130b, and 130c can be located adjacent to one another along one of the plurality of fiber cores). Likewise, each of the other fiber cores (e.g., 122, 124, and 128) can include one or more optical sensors distributed along the length of the respective fiber core. In some embodiments, the plurality of optical sensors 130a, 130b, and 130c are fiber Bragg grating (FBG) sensors. In embodiments in which a plurality of optical sensors (e.g., FBGs) are distributed along a particular fiber core, each FBG may be defined by a unique grating period to allow feedback provided by each FBG to be distinguished from adjacent FBGs. As described above, the plurality of optical sensors 130a, 130b, and 130c can be utilized for different functions. For example, one or more of the optical sensors 130a, 130b, and 130c can be used for at least one of the following: force sensing, shape sensing, and temperature sensing.

While the body can be a tubular body, such as an elongate shaft or a portion thereof, as depicted in FIG. 1A, the body can have any other geometry and/or cross-sectional shape. For example, FIGS. 1B to 1G provide non-limiting examples of other geometries and/or cross-sectional shapes. In particular, a body with a square cross-sectional shape is shown in FIG. 1B, a body with a rectangular cross-sectional shape is shown in FIG. 1C, a body with a polygonal cross-sectional shape is shown in FIG. 1D, a body with a triangular cross-sectional shape is shown in FIG. 1E, a body with an elliptical cross-sectional shape is shown in FIG. 1F, and a body with an oblong cross-sectional shape is shown in FIG. 1G. Each of the bodies depicted in FIGS. 1A to 1G can be elongated and/or flexible. The materials used to form the bodies depicted in FIGS. 1A to 1G are not particularly limited and can include any of a variety of materials. In some embodiments, the bodies can include a hypo tube, a polymer body, or a coil body. For example, the bodies can include materials such as PEBAX®, Nylon, and polyurethane, or the bodies can be constructed of, or incorporate, a metal wire braid.

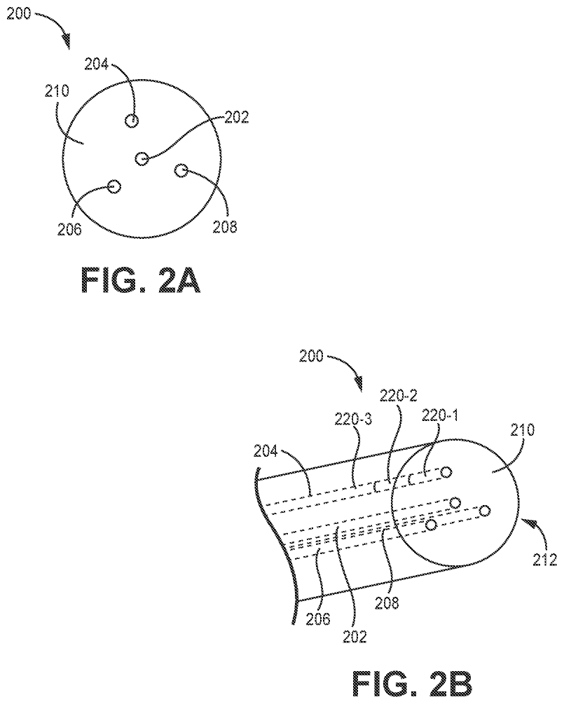

FIG. 2A is an end view and FIG. 2B is an isometric view of a multi-core fiber 200, such as the multi-core fiber 120 depicted in FIG. 1A, in accordance with various embodiments of the invention. The multi-core fiber 200 can include a multi-core fiber body 210 and a plurality (e.g., four) of individual fiber cores 202, 204, 206, and 208 extending along a portion of the multi-core fiber 200. The fiber cores 204, 206, and 208 can be located along an outer circumference of the multi-core fiber 200 and spaced equidistantly from one another (e.g., 120° apart). The fiber core 202 can be a central fiber core. One or more of the fiber cores 202, 204, 206, and 208 can include one or more optical sensors. For example, in the embodiment shown in FIG. 2B, the fiber core 204 includes a plurality of FBGs 220-1, 220-2, and 220-3, wherein the plurality of FBGs 220-1, 220-2, and 220-3 can be located adjacent or approximately adjacent to one another along a portion of the fiber core 204. In embodiments in which the plurality of FBGs 220-1, 220-2, and 220-3 are adjacent to one another, the plurality of FBGs 220-1, 220-2, and 220-3 can be stacked end-to-end, with little to no gap between the adjacent FBGs 220-1, 220-2, and 220-3. The other fiber cores 202, 206, and/or 208 may similarly include one or more optical sensors. For example, a plurality of FBGs (not shown in FIGS. 2A-2B) can be distributed along a length of one or more of the fiber cores 202, 206, and 208. Although four individual fiber cores 202, 204, 206, and 208 are shown in FIGS. 2A-2B, in other embodiments, fewer individual fiber cores can be used, or more individual fiber cores can be used. For example, in some embodiments, only three fiber cores 204, 206, and 208 are used. In other embodiments, additional fiber cores (e.g., a total of six) can be spaced around an outer circumference of the multi-core fiber 200 with an optional center fiber core. In further embodiments, additional fiber cores are contemplated.

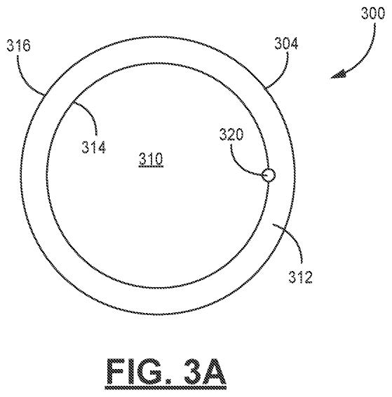

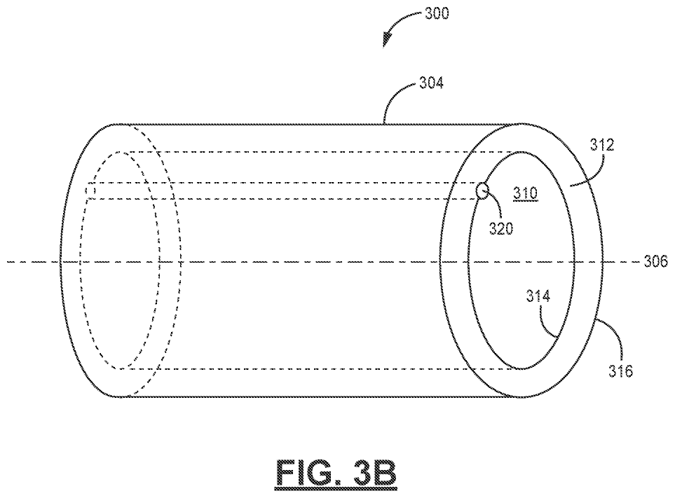

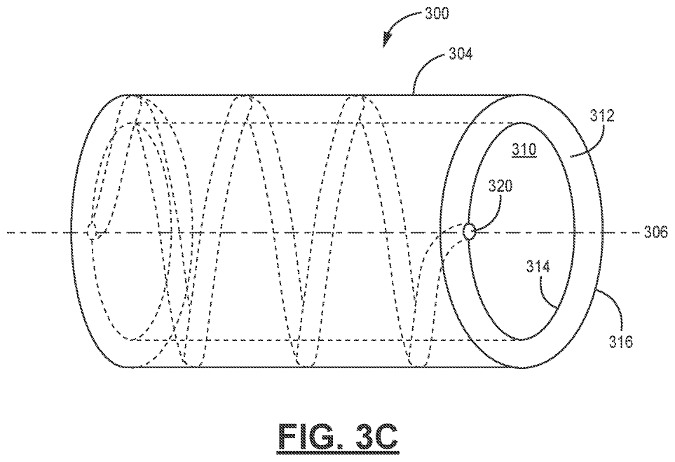

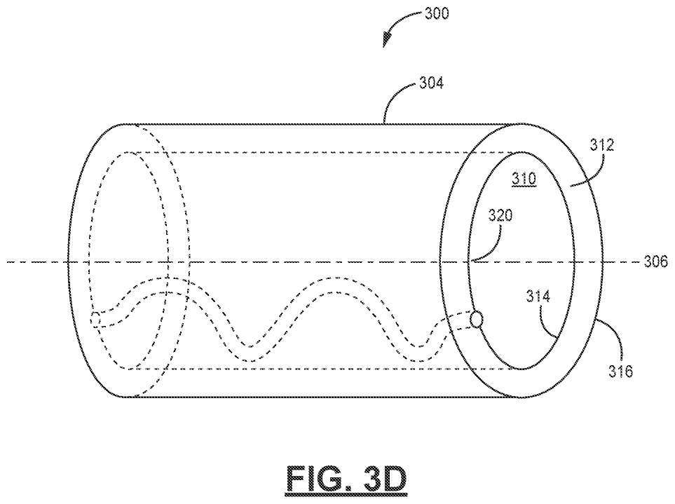

Referring now to FIG. 3A, an end view of a medical device portion 300 is shown with a multi-core fiber partially embedded in a wall of a tubular body, in accordance with one or more embodiments of the invention. As shown in FIG. 3A, the medical device portion 300 includes a tubular body 304 and a multi-core fiber 320 extending along at least a portion of the length of the tubular body 304. The tubular body 304 defines a central longitudinal axis 306 (not shown in FIG. 3A) and a lumen 310. In addition, the tubular body 304 includes an annular wall 312 having an inner circumferential surface 314 and an outer circumferential surface 316. The tubular body 304 can be included in any medical device or medical device portion 300. For example, the tubular body 304 can be included in an ablation catheter, a mapping catheter, a sheath, a guidewire, or an introducer.

In the illustrated embodiment shown in FIG. 3A, the multi-core fiber 320 is at least partially disposed in the annular wall 312 of the tubular body 304 and/or at least partially disposed in the lumen 310 of the tubular body 304. The multi-core fiber 320 in this configuration can at least partially extend into any of various portions of medical devices. For example, the multi-core fiber 320 can at least partially extend into a hoop, a spline of a basket tip, a spline of an array tip, or an ablation tip of a catheter. Although not shown in FIG. 3A, the multi-core fiber 320 can include a plurality of fiber cores, wherein one or more of the plurality of fiber cores can include one or more optical sensors. For example, in some embodiments, the multi-core fiber 320 includes the multi-core fiber 200 from FIGS. 2A-2B discussed above. The one or more optical sensors can be located at one or more locations along a length of each fiber core and may include FBGs. For example, in some embodiments, a plurality of FBGs can be distributed along a length of one or more fiber cores. In some embodiments, one or more of the plurality of fiber cores (including one or more optical sensors) can be used for different functions. For example, in some embodiments, one or more of the plurality of fiber cores of the multi-core fiber 320 is used for at least one of the following: force sensing, shape sensing, and temperature sensing.

Referring now to FIGS. 3B-3D, isometric views of the medical device portion 300 are shown with the multi-core fiber 320 in a parallel configuration (FIG. 3B) and various nonparallel configurations (FIGS. 3C-3D), in accordance with one or more embodiments of the invention. In each of the illustrated embodiments shown in FIGS. 3B-3D, the entire length of the multi-core fiber 320 is at least partially disposed in the annular wall 312 of the tubular body 304. In FIG. 3B, the multi-core fiber 320 is shown in a parallel configuration in which a central longitudinal axis of the multi-core fiber 320 is parallel or substantially parallel to the central longitudinal axis 306 of the tubular body 304, in accordance with some embodiments of the invention. In FIG. 3C, the multi-core fiber 320 is shown in a nonparallel configuration in which the multi-core fiber 320 is helically wound about the central longitudinal axis 306 of the tubular body 304, in accordance with some embodiments of the invention. In FIG. 3D, the multi-core fiber 320 is shown in another nonparallel configuration in which the multi-core fiber 320 is disposed in a serpentine configuration, in accordance with some embodiments of the invention.

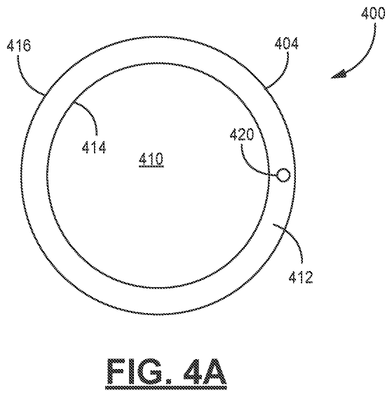

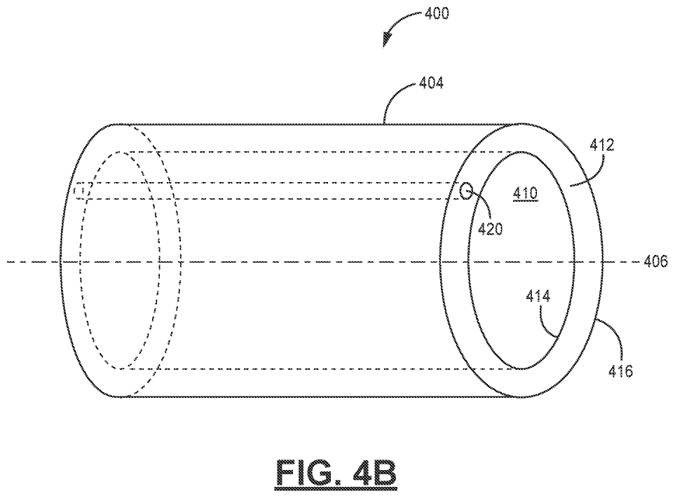

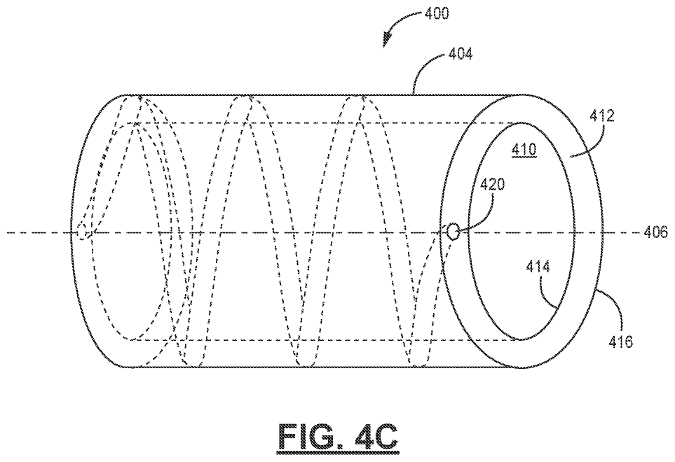

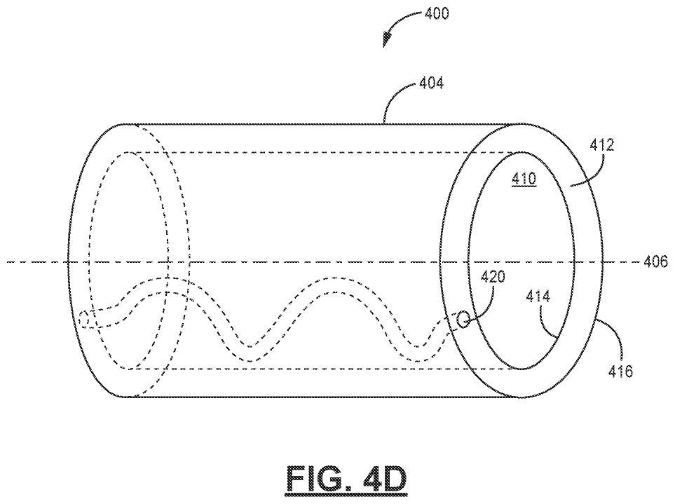

Referring now to FIG. 4A, an end view of a medical device portion 400 is shown with a multi-core fiber completely embedded in a wall of a tubular body, in accordance with one or more embodiments of the invention. As shown in FIG. 4A, the medical device portion 400 includes a tubular body 404 and a multi-core fiber 420 extending along at least a portion of the length of the tubular body 404. The tubular body 404 defines a central longitudinal axis 406 (not shown in FIG. 4A) and a lumen 410. In addition, the tubular body 404 includes an annular wall 412 having an inner circumferential surface 414 and an outer circumferential surface 416. The tubular body 404 can be included in any medical device or medical device portion 400. For example, the tubular body 404 can be included in an ablation catheter, a mapping catheter, a sheath, a guidewire, or an introducer.

In the illustrated embodiment shown in FIG. 4A, the multi-core fiber 420 is entirely disposed in the annular wall 412 of the tubular body between the inner circumference surface 414 and the outer circumferential surface 416 and thus not exposed to either the lumen 410 and/or external environment. The multi-core fiber 420 in this configuration can at least partially extend into any of various portions of medical devices. For example, the multi-core fiber 420 can at least partially extend into a hoop, a spline of a basket tip, a spline of an array tip, or an ablation tip of a catheter. Although not shown in FIG. 4A, the multi-core fiber 420 can include a plurality of fiber cores, wherein one or more of the plurality of fiber cores can include one or more optical sensors. For example, in some embodiments, the multi-core fiber 420 includes the multi-core fiber 200 from FIGS. 2A-2B discussed above. The one or more optical sensors can be located at one or more locations along a length of each fiber core and may include FBGs. For example, in some embodiments, a plurality of FBGs can be distributed along a length of one or more fiber cores. In some embodiments, one or more of the plurality of fiber cores (including one or more optical sensors) can be used for different functions. For example, in some embodiments, one or more of the plurality of fiber cores of the multi-core fiber 420 is used for at least one of the following: force sensing, shape sensing, and temperature sensing.

Referring now to FIGS. 4B-4D, isometric views of the medical device portion 400 are shown with the multi-core fiber 420 in a parallel configuration (FIG. 4B) and various nonparallel configurations (FIGS. 4C-4D), in accordance with one or more embodiments of the invention. In each of the illustrated embodiments shown in FIGS. 4B-4D, the entire length of the multi-core fiber 420 is entirely disposed in the annular wall 412 of the tubular body 404 between the inner circumferential surface 414 and the outer circumferential surface 416. In FIG. 4B, the multi-core fiber 420 is shown in a parallel configuration in which a central longitudinal axis of the multi-core fiber 420 is parallel or substantially parallel to the central longitudinal axis 406 of the tubular body 404, in accordance with some embodiments of the invention. In FIG. 4C, the multi-core fiber 420 is shown in a nonparallel configuration in which the multi-core fiber 420 is helically wound about the central longitudinal axis 406 of the tubular body 404, in accordance with some embodiments of the invention. In FIG. 4D, the multi-core fiber 420 is shown in another nonparallel configuration in which the multi-core fiber 420 is disposed in a serpentine configuration, in accordance with some embodiments of the invention.

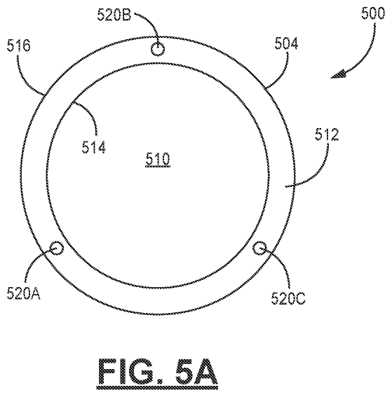

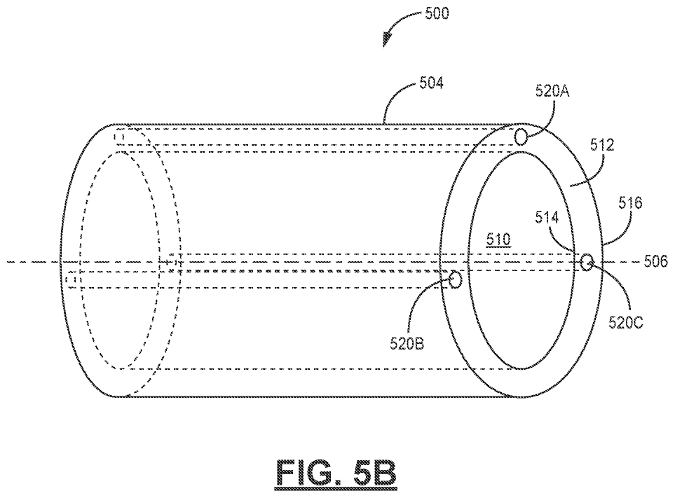

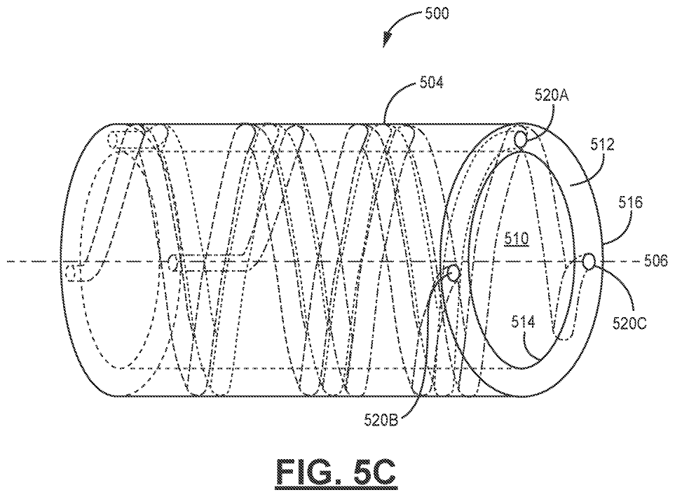

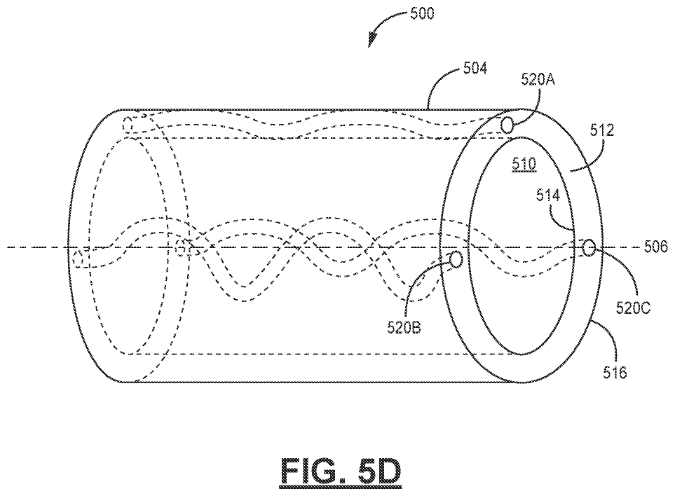

Referring now to FIG. 5A, an end view of a medical device portion 500 is shown with a plurality of single-core fibers completely embedded in a wall of a tubular body, in accordance with one or more embodiments of the invention. As shown in FIG. 5A, the medical device portion 500 includes a tubular body 504 and a plurality (e.g., three) of single-core fibers 520A, 520B, and 520C extending along at least a portion of the length of the tubular body 504. The tubular body 504 defines a central longitudinal axis 506 (not shown in FIG. 5A) and a lumen 510. In addition, the tubular body 504 includes an annular wall 512 having an inner circumferential surface 514 and an outer circumferential surface 516. The tubular body 504 can be included in any medical device or medical device portion 500. For example, the tubular body 504 can be included in an ablation catheter, a mapping catheter, a sheath, a guidewire, or an introducer.

In the illustrated embodiment shown in FIG. 5A, the plurality of single-core fibers 520A, 520B, and 520C are each entirely disposed in the annular wall 512 of the tubular body 504 between the inner circumference surface 514 and the outer circumferential surface 516 and thus not exposed to either the lumen 510 and/or external environment. The plurality of single-core fibers 520A, 520B, and 520C in this configuration can at least partially extend into any of various portions of medical devices. For example, the single-core fibers 520A, 520B, and 520C can at least partially extend into a hoop, a spline of a basket tip, a spline of an array tip, or an ablation tip of a catheter. As shown in FIG. 5A, in some embodiments, the plurality of single-core fibers 520A, 520B, and 520C can be equidistantly spaced apart from one another (e.g., about 120° apart).

Although not shown in FIG. 5A, each of the plurality of single-core fibers 520A, 520B, and 520C includes a single fiber core, wherein each fiber core can include one or more optical sensors. The one or more optical sensors can be located at one or more locations along a length of the single-core fibers 520A, 520B, and 520C and may include FBGs. For example, in some embodiments, a plurality of FBGs can be distributed along a length of one or more of the single-core fibers 520A, 520B, and 520C. Information provided by the one or more optical sensors can be utilized to detect a number of parameters, including force, shape, and/or temperature. In some embodiments, information from optical sensors located on a plurality of different single-core fibers 520A, 520B, and 520C is required to detect one or more parameters. For example, in some embodiments, shape information requires input from optical sensors located in a plurality of different single-core fibers. In this way, the plurality of single-core fibers 520A, 520B, and 520C can provide the same functionality as a multi-core fiber. While three single-core fibers are shown in FIG. 5A, the three single-core fibers are shown as an example. In other embodiments, two or more single-core fibers can be used together to provide the functionality of a multi-core fiber.

Referring now to FIGS. 5B-5D, isometric views of the medical device portion 500 are shown with the plurality of single-core fibers 520A, 520B, and 520C in a parallel configuration (FIG. 5B) and various nonparallel configurations (FIGS. 5C-5D), in accordance with one or more embodiments of the invention. In each of the illustrated embodiments shown in FIGS. 5B-5D, the entire length of each of the plurality of single-core fibers 520A, 520B, and 520C is entirely disposed in the annular wall 512 of the tubular body 504 between the inner circumferential surface 514 and the outer circumferential surface 516. In FIG. 5B, each of the three single-core fibers 520A, 520B, and 520C is shown in a parallel configuration in which a central longitudinal axis of each single-core fiber 520A, 520B, and 520C is parallel or substantially parallel to the central longitudinal axis 506 of the tubular body 504, in accordance with some embodiments of the invention. In FIG. 5C, each of the three single-core fibers 520A, 520B, and 520C is shown in a nonparallel configuration in which each of the three single-core fibers 520A, 520B, and 520C is helically wound about the central longitudinal axis 506 of the tubular body 504, in accordance with some embodiments of the invention. In FIG. 5D, each of the three single-core fibers 520A, 520B, and 520C is shown in another nonparallel configuration in which each of the three single-core fibers 520A, 520B, and 520C is disposed in a serpentine configuration, in accordance with some embodiments of the invention.

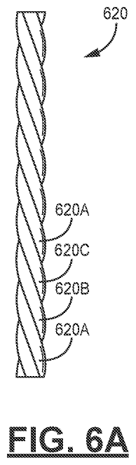

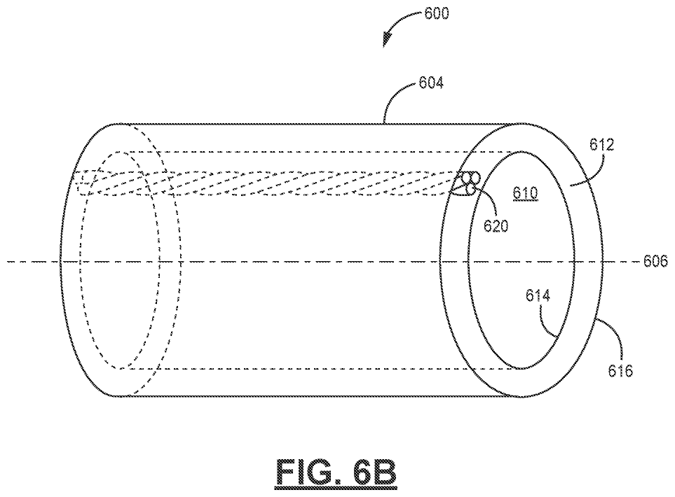

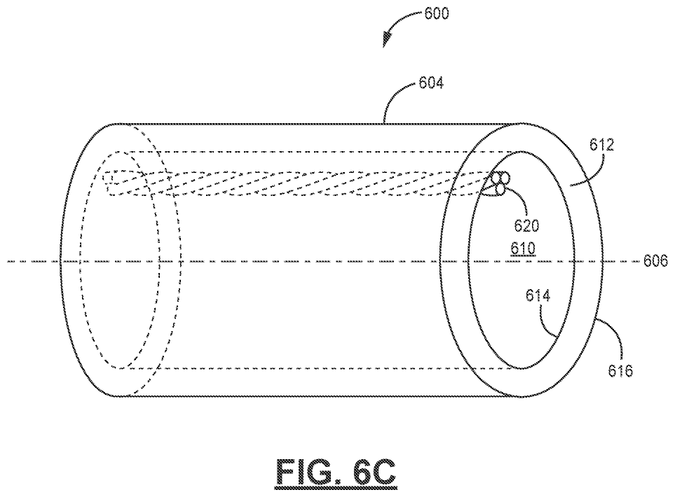

Referring now to FIG. 6A, a side view of a plurality of single-core fibers 620 are shown in a twisted configuration, in accordance with one or more embodiments of the invention. As shown in FIG. 6A, a twisted configuration includes a plurality of single-core fibers 620A, 620B, and 620C helically twisted together. Although not shown in FIG. 6A, each of the plurality of single-core fibers 620A, 620B, and 620C includes a fiber core, wherein each fiber core can include one or more optical sensors. The one or more optical sensors can be located at one or more locations along a length of the single-core fibers 620A, 620B, and 620C and may include FBGs. For example, in some embodiments, a plurality of FBGs can be distributed along a length of one or more of the single-core fibers 620A, 620B, and 620C. The plurality of single-core fibers 620A, 620B, and 620C can be utilized for one or more of force sensing, shape sensing, and/or temperature sensing. In this way, the plurality of single-core fibers 620A, 620B, and 620C can provide the functionality of a multi-core fiber, as described above. While three single-core fibers are shown in a twisted configuration in FIG. 6A, in other embodiments, two or more single-core fibers can be helically twisted together and used to provide the functionality of a multi-core fiber.

Referring now to FIGS. 6B-6C, isometric views of a medical device portion 600 are shown with the plurality of single-core fibers 620A, 620B, and 620C from FIG. 6A, in accordance with one or more embodiments of the invention. As shown in FIGS. 6B-6C, the medical device portion 600 can include a tubular body 604 and the plurality (e.g., three) of single-core fibers 620A, 620B, and 620C can extend in the twisted configuration along at least a portion of the length of the tubular body 604. The tubular body 604 defines a central longitudinal axis 606 and a lumen 610. In addition, the tubular body 604 includes an annular wall 612 having an inner circumferential surface 614 and an outer circumferential surface 616. In FIG. 6B, the plurality of single-core fibers 620A, 620B, and 620C are shown in the twisted configuration and entirely disposed in the annular wall 612 of the tubular body 604 between the inner circumference surface 614 and the outer circumferential surface 616 and thus not exposed to either the lumen 610 and/or external environment. In FIG. 6C, the plurality of single-core fibers 620A, 620B, and 620C are shown disposed in the twisted configuration and disposed in the lumen of the tubular body 604.

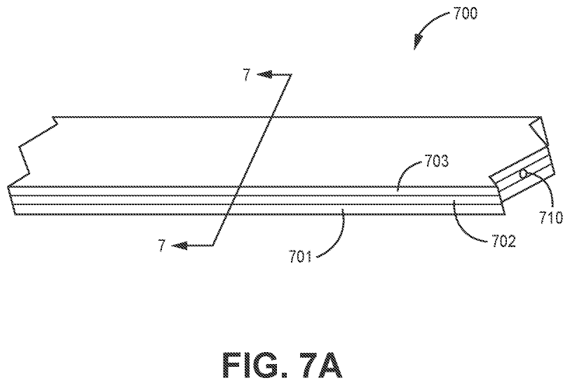

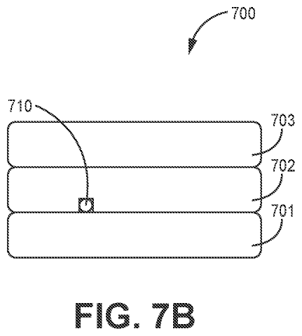

FIGS. 7A-7B are isometric and cross-sectional views of a multi-layered medical device portion in accordance with one or more embodiments of the invention. The portion of the medical device 700 can form any portion of a medical device and thus the medical devices and portions thereof are not particularly limited. In some embodiments, the portion of the medical device 700 can include planar or non-planar, flexible or non-flexible portions of medical devices, such as mapping catheters and/or ablation catheters. For example, in one embodiment, the portion of the medical device 700 can include a portion of an arm or strut of a grid or a planar end of a mapping and/or ablation catheter (e.g., planar array catheters). In another embodiment, the portion of the medical device 700 can include a portion of a spline of a basket electrode assembly of a mapping and/or ablation catheter (e.g., basket catheters).

The medical device portion 700 can generally comprise one or more layers and a multi-core fiber. As illustrated in FIGS. 7A-7B, in one embodiment, the medical device portion 700 can comprise a first layer 701, a second layer 702, and a third layer 703, and a multi-core fiber 710 deposited on a surface of the first layer 701 and/or disposed between the first layer 701 and second layer 702. In another embodiment, the medical device portion 700 can comprise the first layer 701 only, the first layer 701 and second layer 702 only, or one or more additional layers (not shown in FIGS. 7A-7B), which can be provided on a top surface or bottom surface of the first layer 701. In another embodiment, the medical device portion 700 can comprise a plurality of multi-core fibers (not shown in FIGS. 7A-7B). In another embodiment, the multi-core fiber 710 can be deposited on the surface of a layer other than the first layer 701, such as the second layer 702, the third layer 703, and, if present, any of the one or more additional layers. The multi-core fiber 710 can partially or completely extend along the length of the layer on which it is deposited. Although not shown in FIGS. 7A-7B, the multi-core fiber 710 can include a plurality of fiber cores, each fiber core including one or more optical sensors (e.g., FBGs).

The materials forming the first layer 701, second layer 702, and third layer 703, and if present, any of the additional layers are not particularly limited. In some embodiments, the first layer 701, second layer 702, and third layer 703 each independently include at least one of the following layers: a support layer (e.g., a substrate, flexible substrate, or flex printed substrate, such as a flexible printed circuit board or a substrate formed of fiberglass, a non-conductive material, a polymer, or nitinol), conductive layer (e.g., a conductive trace), dielectric layer, insulative layer, electrode (e.g., microelectrode), contact pad, seed layer, or mask. For example, in one embodiment, the first layer 701 can be a substrate and include a multi-core fiber 710 deposited on a substrate surface, the second layer 702 can be deposited on the first layer and can form a dielectric layer, and the third layer 703 can be deposited on the second layer and can form one or more electrodes (e.g., microelectrodes), wherein one or more of the electrodes is optionally electrically coupled to one or more conductive traces (not shown in FIGS. 7A-7B).

The first layer 701, second layer 702, and/or third layer 703 can be a flexible substrate forming a strut, an arm, or a spline of an expandable portion of an ablation catheter or mapping catheter. For example, in some embodiments, the portion of the medical device 700 is a distal end portion of the medical device, with a multi-core fiber 710 extending through a handle assembly (not shown) and/or flexible elongate shaft (not shown) to the distal end portion. The distal end portion can include an expandable portion of an ablation catheter or mapping catheter. The expandable portion can include a flexible substrate 701 (e.g., as the first layer) which forms at least a portion of a strut, an arm, or a spline. The multi-core fiber 710 can be disposed on or in the strut, the arm, or the spline, and can extend partially or completely along the length thereof under the second layer 702 and third layer 703. In some embodiments, the multi-core fiber 710 extends past one or more electrodes, which can form the second layer 702 and/or third layer 703. In other embodiments, the multi-core fiber 710 extends proximal to, but not past, one or more electrodes, which can form the second layer 702 and/or third layer 703.

The layers of the medical device portion 700 can each be independently formed through the same or different additive and/or subtractive manufacturing processes. For example, in some embodiments, all the layers or at least one of the first layer 701, second layer 702, and third layer 703 is a printed layer or printed overlayer. Printing processes can include, without limitation, direct write printing of electronics including aerosol jet, micro-dispensing (micropen), and ink jet printing, as well as screen printing and plating. Other additive processes include chemical vapor deposition and depositing material onto a mold through an additive process. Once the material has been deposited, the material can be cured, and the mold can be released from the material. In other embodiments, one or more of the first layer 701, second layer 702, and third layer 703 can be formed through a subtractive process (e.g., laser etching, chemical etching, machining, etc.).

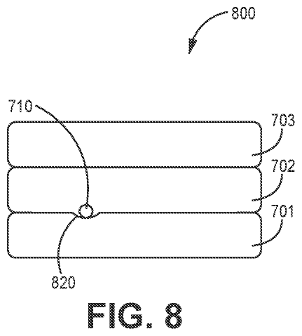

FIG. 8 is a cross-sectional view of the medical device portion 800 taken along line 7-7 to illustrate another embodiment of the invention. As shown in FIG. 8, the medical device portion 800 is similar to the medical device portion 700 depicted in FIGS. 7A and 7B, except the medical device portion 800 further includes a channel 820. In the illustrated embodiment, the channel 820 can be formed in the first layer 701. In other embodiments, the channel 820 can be formed in one or more of the first layer 701, second layer 702, third layer 703, and if present, the one or more additional layers.

The channel 820 can optionally be used for securing or holding in place the multi-core fiber to simplify the manufacturing of the medical devices or portions thereof. In some embodiments, the channel 820 can be shaped to permit some movement of the multi-core fiber 710 within the channel 820. For example, in one embodiment, the channel 820 can be sufficient to keep the multi-core fiber 710 within the channel, but otherwise generally permits some movement (e.g., rolling). In these embodiments, once the multi-core fiber 710 is disposed in the channel 820, the next layer can be deposited thereon to fill the unoccupied space in the channel layer 820; or a filler material, additive material, or intermediate layer can be locally deposited in, near, and/or around the channel layer 820, partially or completely coating the multi-core fiber 710, prior to depositing the next layer, to fill the unoccupied space in the channel layer 820. In other embodiments, the channel 820 can be shaped to permit no movement of the multi-core fiber 710. For example, in one embodiment, the channel 820 can snap or lock the multi-core fiber 710 in place.

The channel 820 can be formed by casting precursor materials or by pressing an initially planar material, among other methods. In some embodiments, the channel 820 can be formed in a deformable material. For example, the channel 820 can be formed in a metal, polymer, or other type of material, which can be deformed via application of heat and/or pressure to the material. In one embodiment, if the channel 820 is formed in a layer comprising a metal, the channel 820 can be formed by casting the metal and/or pressing the metal (e.g., via tool and die) to form the channel 820 in the layer. In some embodiments where the channel 820 is formed in a layer comprising a polymer, the polymer can be formed such that the channel 820 is formed in the layer. For example, the polymer can be cast such that the layer includes the channel 820 and/or the polymer can be heated and/or pressure can be applied to the polymer to form the channel 820.

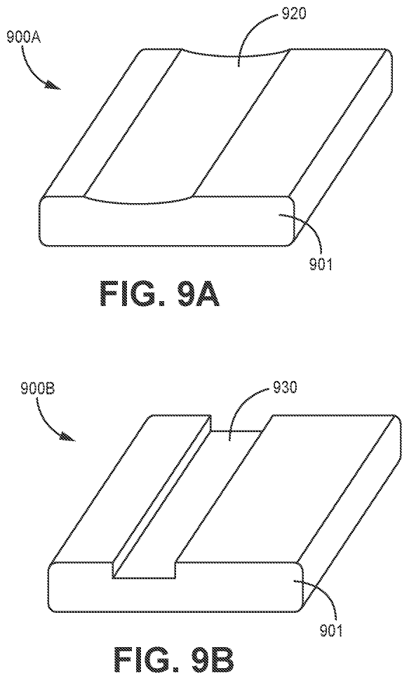

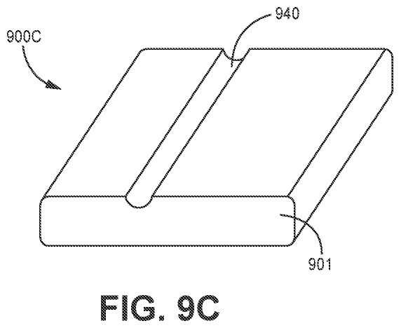

FIGS. 9A, 9B, and 9C are isometric views illustrating other configurations of channels 900A, 900B, and 900C, respectively. FIG. 9A depicts a channel 920 formed in a layer 901, which can be any of the layers described above. The channel 920 has a rounded base that permits some movement or rolling of a multi-core fiber (not shown in FIG. 9A) within the channel 920, but otherwise keeps the multi-core fiber within the channel 920 (e.g., to prevent it from rolling off). This embodiment can be simple to manufacture as it does not require precise measurements or fabrication techniques.

FIG. 9B depicts a channel 930 formed in the layer 901, which can similarly be any of the layers described above. The channel 930 has a square-like or oblong-like base that permits some movement or rolling of a multi-core fiber (not shown in FIG. 9B) within the channel 920, but otherwise keeps the multi-core fiber within the channel 920 (e.g., to prevent it from rolling off). This embodiment can be simple to manufacture as it does not require precise measurements or fabrication techniques.

FIG. 9C depicts a channel 940 formed in the layer 901, which can similarly be any of the layers described above. The channel 940 has a form-fitting rounded base that can snap or lock a multi-core fiber (not shown in FIG. 9C) in place, permitting minimal to no movement within the channel 940. This embodiment can be used for complex medical devices or medical devices requiring a multi-core fiber to be precisely located. Discussion of Possible Embodiments

The following includes non-exhaustive descriptions of possible embodiments of the present invention.

According to some aspects, a medical device may include a tubular body defining a central longitudinal axis and a lumen, the tubular body including an annular wall having an inner circumferential surface and an outer circumferential surface; and a multi-core fiber extending along at least a portion of the length of the tubular body, wherein the multi-core fiber is at least partially disposed in the annular wall of the tubular body.

The medical device of the preceding paragraph may optionally include, additionally and/or alternatively, any one or more of the following features, configurations, and/or additional components.

For example, in certain aspects, the multi-core fiber is entirely disposed in the annular wall of the tubular body between the inner circumferential surface and the outer circumferential surface.

In certain aspects, the multi-core fiber defines a central longitudinal axis that is nonparallel to the central longitudinal axis of the tubular body.

In certain aspects, the multi-core fiber is helically wound about the central longitudinal axis of the tubular body.

In certain aspects, the multi-core fiber is disposed in a serpentine configuration.

In certain aspects, the multi-core fiber includes a plurality of fiber cores, each fiber core including one or more fiber Bragg gratings distributed along the length of the fiber core.

In certain aspects, one or more fiber cores of the multi-core fiber is used for at least one of the following: force sensing, shape sensing, and temperature sensing.

In certain aspects, the tubular body is included in an ablation catheter, a mapping catheter, a sheath, a guidewire, or an introducer.

In certain aspects, the multi-core fiber at least partially extends into a hoop, a spline of a basket tip, a spline of an array tip, or an ablation tip of a catheter.

According to further aspects, a medical device may include a tubular body defining a central longitudinal axis and a lumen, the tubular body including an annular wall having an inner circumferential surface and an outer circumferential surface; and a plurality of single-core fibers, each single-core fiber extending along at least a portion of the length of the tubular body and defining a central longitudinal axis that is nonparallel to the central longitudinal axis of the tubular body.

The medical device of the preceding paragraph may optionally include, additionally and/or alternatively, any one or more of the following features, configurations, and/or additional components.

For example, in certain aspects, each of the plurality of single-core fibers is helically wound about the central longitudinal axis of the tubular body.

In certain aspects, each of the plurality of single-core fibers is disposed in a serpentine configuration.

In certain aspects, each of the plurality of single-core fibers are helically twisted together.

In certain aspects, the plurality of single-core fibers are disposed in the lumen of the tubular body.

In certain aspects, the plurality of single-core fibers are entirely disposed in the annular wall of the tubular body between the inner circumferential surface and the outer circumferential surface.

In certain aspects, the plurality of single-core fibers includes three single-core fibers spaced 120 degrees apart.

In certain aspects, each of the plurality of single-core fibers includes a fiber core, each fiber core including one or more fiber Bragg gratings distributed along the length of the fiber core.

According to further aspects, a medical device may include a multi-core fiber disposed on a top surface of a first layer and extending along at least a portion of the length of the first layer, wherein the multi-core fiber includes a plurality of fiber cores, each fiber core including one or more fiber Bragg gratings distributed along the length of the fiber core; and a second layer deposited or printed on the first layer.

The medical device of the preceding paragraph may optionally include, additionally and/or alternatively, any one or more of the following features, configurations, and/or additional components.

For example, in certain aspects, a channel is formed in the first layer and the multi-core fiber is disposed in the channel.

In certain aspects, the first layer is a flexible substrate forming an arm, a strut, or a spline of an expandable portion of an ablation catheter or a mapping catheter; and wherein the second layer comprises at least one of the following: a conductor, a dielectric, an insulator, and an electrode.

Embodiments are described herein of various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it may be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment(s) is included in at least one embodiment. Thus, appearances of the phrases “in various embodiments,” “in some embodiments,” “in one embodiment,” or “in an embodiment,” or the like, in places throughout the specification, are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

What is claimed is:

1. A medical device comprising:
a tubular body defining a central longitudinal axis and a lumen, the tubular body including an annular wall having an inner circumferential surface and an outer circumferential surface; and
a multi-core fiber extending along at least a portion of the length of the tubular body, wherein the multi-core fiber is at least partially disposed in the annular wall of the tubular body.

2. The medical device of claim 1, wherein the multi-core fiber is entirely disposed in the annular wall of the tubular body between the inner circumferential surface and the outer circumferential surface.

3. The medical device of claim 1, wherein the multi-core fiber defines a central longitudinal axis that is nonparallel to the central longitudinal axis of the tubular body.

4. The medical device of claim 1, wherein the multi-core fiber is helically wound about the central longitudinal axis of the tubular body.

5. The medical device of claim 1, wherein the multi-core fiber is disposed in a serpentine configuration.

6. The medical device of claim 1, wherein the multi-core fiber includes a plurality of fiber cores, each fiber core including one or more fiber Bragg gratings distributed along the length of the fiber core.

7. The medical device of claim 6, wherein one or more fiber cores of the multi-core fiber is used for at least one of the following: force sensing, shape sensing, and temperature sensing.

8. The medical device of claim 1, wherein the tubular body is included in an ablation catheter, a mapping catheter, a sheath, a guidewire, or an introducer.

9. The medical device of claim 1, wherein the multi-core fiber at least partially extends into a hoop, a spline of a basket tip, a spline of an array tip, or an ablation tip of a catheter.

10. A medical device comprising:
a tubular body defining a central longitudinal axis and a lumen, the tubular body including an annular wall having an inner circumferential surface and an outer circumferential surface; and
a plurality of single-core fibers, each single-core fiber extending along at least a portion of the length of the tubular body and defining a central longitudinal axis that is nonparallel to the central longitudinal axis of the tubular body.

11. The medical device of claim 10, wherein each of the plurality of single-core fibers is helically wound about the central longitudinal axis of the tubular body.

12. The medical device of claim 10, wherein each of the plurality of single-core fibers is disposed in a serpentine configuration.

13. The medical device of claim 10, wherein the plurality of single-core fibers are helically twisted together.

14. The medical device of claim 13, wherein the plurality of single-core fibers are disposed in the lumen of the tubular body.

15. The medical device of claim 10, wherein the plurality of single-core fibers are entirely disposed in the annular wall of the tubular body between the inner circumferential surface and the outer circumferential surface.

16. The medical device of claim 10, wherein the plurality of single-core fibers includes three single-core fibers spaced 120 degrees apart.

17. The medical device of claim 10, wherein each of the plurality of single-core fibers includes a fiber core, each fiber core including one or more fiber Bragg gratings distributed along the length of the fiber core.

18. A medical device comprising:
a multi-core fiber disposed on a top surface of a first layer and extending along at least a portion of the length of the first layer, wherein the multi-core fiber includes a plurality of fiber cores, each fiber core including one or more fiber Bragg gratings distributed along the length of the fiber core; and
a second layer deposited or printed on the first layer.

19. The medical device of claim 18, wherein a channel is formed in the first layer and the multi-core fiber is disposed in the channel.

20. The medical device of claim 18, wherein the first layer is a flexible substrate forming an arm, a strut, or a spline of an expandable portion of an ablation catheter or a mapping catheter; and wherein the second layer comprises at least one of the following: a conductor, a dielectric, an insulator, and an electrode.
